(19) United States
(12) Patent Application Publication
(10) Pub. No.: US 2006/0140915 A1
(43) Pub. Date: Jun. 29, 2006

(54) Veterinary protocol for cellular regeneration

(76) Inventors: Richard A. Schatz, Rancho Santa Fe, CA (US); William H. D. de Burgh, Rancho Santa Fe, CA (US)

Correspondence Address: NEIL K. NYDEGGER;NYDEGGER & ASSOCIATES, 348 Olive Street, San Diego, CA, 92103, US

(21) Appl. No.: 11/024,188

(22) Filed: Dec. 28, 2004

(51) Int. Cl. A61K 35/12

(52) U.S. Cl. 424/93.7

(57) ABSTRACT

A medicament and corresponding treatment protocol for in-vivo cellular regeneration within a mammal is described. Applications include the treatment of injuries to bone, cartilage, ligaments and tendons in humans and animals. Treatment includes the step of administering one or more therapeutically effective doses of medicament into an injured, living mammal. For the treatment, the medicament includes a plurality of stem cells and an angiogenic factor such as vascular endothelial growth factor (VEGF). In one implementation, non-embryonic stem cells are harvested from the mammal after injury for use in the medicament. Once harvested, the stem cells are typically concentrated (by removing non-stem cells from the harvested cell population) in a laboratory and then mixed with angiogenic factor to prepare the medicament. In an exemplary procedure, approximately ten therapeutically effective doses are injected into the body at or near the injured tissue during a healing period following the injury.

### FIELD OF THE INVENTION

The present invention pertains generally to medicaments and therapeutic treatment methods for repairing injured tissue. More particularly, the present invention pertains to processes which regenerate cellular tissue and blood vessels in mammals. The present invention is particularly, but not exclusively, useful as a medicament and corresponding therapeutic dosing regimen for healing injured bone, cartilage, ligaments and tendons.

### BACKGROUND OF THE INVENTION

Injuries to bone, cartilage, ligaments and tendons occur all too often in humans and animals, and are among the most challenging injuries to effectively treat. These injuries can include stress fractures, tears, hyperextension and, in some cases, detachment or separation of the effected bone, cartilage, ligament or tendon from surrounding tissues. Injuries such as these are often accompanied by pain, reduced function including a decreased range of motion, and can result in joint instability.

The body's natural response to these types of injury typically includes three distinct phases. The first phase, which typically lasts several days, can be characterized as an inflammatory response that includes swelling, soreness, heat and pain. In general, the inflammatory response signals the body to trigger the second phase in which healing occurs. During this second phase, cells are regenerated to create the new tissue that is necessary to repair or replace the injured tissue. Essential precursors for this natural healing process typically include stem cells (i.e. undifferentiated or partially differentiated cells), one or more growth factors and an ample supply of blood.

During the natural healing process, the required ingredients (e.g. stem cells, blood, etc.) must be transported to the affected, damaged area from other areas of the body where they are generated or stored. Thus, inadequate circulation at and near the damaged area can significantly delay or reduce the beneficial effects of healing. As a direct result of this phenomena, muscle and skin tissues, which have a relatively vast circulatory system, tend to naturally heal much quicker and to a greater extent than ligaments and tendons which typically have only minimal circulation within their tissue structure.

In addition to the inflammation and healing phases, the body's natural response to an injury also includes a third phase in which scar tissue is formed to stabilize the injured area. In general, the formation of scar tissue tends to interrupt healing and can often result in undesirable consequences. For example, scar tissue can decrease the elasticity of ligaments and tendons, reducing their performance and increasing the likelihood that the affected area is subsequently re-injured. Scar tissue can also reduce local circulation, which in turn, can have an adverse effect on healing.

With the above in mind, treatment approaches have generally focused on augmenting the body's natural healing response while reducing scarring and the adverse effects of inflammation. In one technique, stem cells and growth factor have been harvested from bone marrow and injected into the general vicinity of the injury. While this practice has been somewhat efficacious for certain types of injured tissue (e.g. muscle tissues), it has produced less than optimal results when used to treat injured bone, cartilage, ligaments and tendons. Specifically, for these later applications, insufficient local circulation has, by and large, prevented the migration of the necessary healing components (i.e. stem cells, blood, etc.) to the locations within the injured structure (i.e. tendon, ligament, etc.) where they are needed.

In light of the above, it is an object of the present invention to provide medicaments and corresponding therapeutic methods suitable for the purposes of augmenting a body's natural healing process. It is still another object of the present invention to provide medicaments and corresponding therapeutic methods for treating injured bone, cartilage, ligaments and tendons. Yet another object of the present invention is to provide medicaments and corresponding therapeutic methods for treating injuries that are easy to use, relatively simple to implement, and comparatively cost effective.

### SUMMARY OF THE INVENTION

The present invention is directed to a medicament and corresponding treatment protocol for in-vivo cellular regeneration within a mammal. Typical applications include, but are not limited to, the treatment of injured bone, cartilage, ligaments and tendons in humans and animals. For the present invention, the treatment method includes the step of administering into the living mammal a therapeutically effective dose of medicament having a plurality of stem cells and an angiogenic factor.

For the present methods, the stem cells can be harvested from the mammal requiring treatment and can be embryonic or non-embryonic stem cells. More specifically, as used herein, the term “stem cell” and its derivatives is intended to include both undifferentiated and partially differentiated cells. Moreover, the present disclosure should not be interpreted to preclude the use of stem cells that have been modified or genetically altered. In addition to embryonic sources, these stem cells can be harvested from mammals of all ages and, if properly stored, are usable indefinitely. Typically, the stem cells are harvested from bodily portions that are easily accessible and rich in stem cells. These portions can include adipose tissues (i.e. fatty tissues), bone marrow and other tissues known in the pertinent art for harvesting stem cells. Once harvested, the stem cells are, in most cases, concentrated in-vitro (by removing non-stem cells from the harvested cell population) and then combined with angiogenic factor to prepare the medicament. In some implementations, the stem cells are mixed with the angiogenic factor prior to administration. In other cases, the stem cells and angiogenic factor are introduced into the mammal separately.

As used herein, the term “angiogenic factor” and its derivatives means any protein, polypeptide, mutein or portion thereof that is capable of, directly or indirectly, inducing blood vessel growth. Examples of angiogenic factor include, but are not limited to, acidic and basic fibroblast growth factors (aFGF and bFGF), vascular endothelial growth factor (VEGF-1), (VEGF165), epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage-CSF (M-CSF), granulocyte/macrophage CSF (GM-CSF), angiopoetin-1 (Ang1) and nitric oxidesynthase (NOS); as well as muteins or fragments of a mitogen capable of inducing or promoting blood vessel growth. For the invention, the angiogenic factor can be introduced into the living mammal as a protein. Alternatively, the angiogenic factor can be introduced into the living mammal as a DNA plasmid (e.g. a single strand of DNA which directs other cells to produce the angiogenic factor as a protein). This DNA plasmid can be introduced using a vector such as a viral vector, a liposome vector or an ultrasound vector.

In accordance with the present methods, one to ten therapeutically effective doses are typically administered during a healing period subsequent to the injury. For each dose, the medicament is typically applied directly to the tissue (i.e. topically) or injected into the body at or near the injured tissue. For example, in one implementation, the medicament is applied directly to tissue using a bio-absorbable polymer mesh that has been loaded with stem cells and angiogenic factor. Injections can be made using a syringe to administer the medicament directly into the tissue, intra-muscular, intravenously, intra-arterially or, in some cases, transdermally.

### DESCRIPTION OF THE PREFERRED EMBODIMENTS

A treatment protocol for the in vivo regeneration of cellular tissue to repair or replace injured tissue begins with the preparation of a medicament. For purposes of the present discussion, the treatment of a horse having a bowed tendon will be discussed with the understanding that the present methods are not limited to one specific type of mammal, tissue or injury. Instead, those skilled in the pertinent art will quickly appreciate that the present methods can be used for the treatment of all mammals, including humans, and can be used to treat different types of injuries (including detectable and undetectable injuries) and different types of tissues.

Continuing with the example, to prepare a suitable medicament, adipose (i.e. fatty) tissue is surgically harvested from the subject mammal at a location wherein removal of such tissue will least affect the mammal. For the horse, a suitable location to extract adipose tissue is near the tail head. The harvested sample generally includes a cell population having stem cells and non-stem cells (e.g. fatty cells). Once extracted, the sample is processed in a sterile laboratory environment to remove some or all of the fatty cells and thereby concentrate the stem cells. Next, the concentrated stem cells are combined (e.g. mixed) with angiogenic factor or plasmid, such as vascular endothelial growth factor (VEGF).

Once combined, the medicament may be apportioned into approximately ten therapeutically effective doses or maintained as a single dose. If apportioned, each dose typically includes between approximately 200 mcg and approximately 100 mg vascular endothelial growth factor (VEGF) and between approximately 5 million and approximately 500 million stem cells. Once apportioned, the doses are loaded into injection syringes.

In one exemplary implementation of the treatment method, the ten doses are administered to the subject horse over a period of time following the injury, with one dose administered every two days. More specifically, each dose is injected into the affected leg of the horse at or near the bowed tendon. Typically, the present methods are used in conjunction with standard post-injury splinting, physical therapy and rehab procedures.

While the particular Veterinary Protocol For Cellular Regeneration and corresponding methods of use as herein shown and disclosed in detail are fully capable of obtaining the objects and providing the advantages herein before stated, it is to be understood that they are merely illustrative of the presently preferred embodiments of the invention and that no limitations are intended to the details of construction or design herein shown other than as described in the appended claims.

What is claimed is:

1. A method for in-vivo cellular regeneration in a mammal, said method comprising the step of administering a therapeutically effective dose of medicament into the living mammal, said medicament including a plurality of stem cells and an angiogenic factor.

2. A method as recited in claim 1 wherein said angiogenic factor is a vascular endothelial growth factor (VEGF).

3. A method as recited in claim 1 wherein said stem cells are harvested from the living mammal.

4. A method as recited in claim 3 wherein said stem cells are harvested from adipose tissue.

5. A method as recited in claim 1 wherein said stem cells are non-embryonic stem cells.

6. A method as recited in claim 1 wherein said medicament is administered to regenerate bone tissue.

7. A method as recited in claim 1 wherein said medicament is administered to regenerate cartilage tissue.

8. A method as recited in claim 1 wherein said medicament is administered to regenerate ligament tissue.

9. A method as recited in claim 1 wherein said medicament is administered to repair injured tissue.

10. A method as recited in claim 9 wherein said medicament is injected into the injured tissue.

11. A method as recited in claim 1 wherein said medicament is administered intramuscularly.

12. A method as recited in claim 1 wherein said medicament is administered intravenously.

13. A method as recited in claim 1 wherein said medicament is administered intra-arterially.

14. A method as recited in claim 1 wherein said mammal is a horse.

15. A method as recited in claim 1 wherein said plurality of stem cells is introduced into the living mammal separately from said angiogenic factor.

16. A method as recited in claim 1 wherein said angiogenic factor is introduced into the living mammal as a protein.

17. A method as recited in claim 1 wherein said angiogenic factor is introduced into the living mammal as a DNA plasmid.

18. A method as recited in claim 17 wherein said DNA plasmid is introduced using a vector selected from the group of vectors consisting of a viral vector, a liposome vector and an ultrasound vector.

19. A method as recited in claim 1 wherein said medicament is administered topically.

20. A method as recited in claim 19 wherein said medicament is applied directly to tissue using a bio-absorbable polymer mesh loaded with said stem cells and said angiogenic factor.

21. A method for in-vivo cellular regeneration in a mammal, said method comprising the steps of:
harvesting a population of cells from said living mammal, said population having stem cells and non-stem cells;
removing a portion of the non-stem cells from said cell population to produce a concentrated cell population; and
introducing said concentrated cell population and an angiogenic factor into said living mammal to regenerate tissue therein.

22. A method as recited in claim 21 further comprising the steps of:
mixing said concentrated cell population and said angiogenic factor to establish a mixture; and
apportioning the mixture into a plurality of therapeutically effective doses, each said dose being efficacious to regenerate a target type of tissue cell.

23. A method as recited in claim 22 wherein each said dose includes between approximately 200 mcg and approximately 100 mg vascular endothelial growth factor (VEGF) and between approximately 5 million and approximately 500 million stem cells.

24. A method as recited in claim 22 wherein said plurality of therapeutically effective doses is in a range of two to ten doses.

25. A system for in-vivo cellular regeneration in a mammal, said system comprising:
a means for surgically harvesting a population of cells from said living mammal;
a means for removing a portion of non-stem cells from said cell population to produce a concentrated cell population; and
a means for introducing said concentrated cell population and an angiogenic factor into said living mammal to regenerate tissue therein.

26. A system as recited in claim 25 wherein said introducing means comprises a syringe.

27. A medicament for in-vivo cellular regeneration in a living mammal, said medicament comprising:
a plurality of stem cells harvested from said living mammal; and
a vascular endothelial growth factor (VEGF).
